(19) United States
(12) Patent Application Publication
(10) Pub. No.: US 2024/0033671 A1
(43) Pub. Date: Feb. 1, 2024

(54) COMPLEX AIR PURIFIER HAVING STERILIZATION FUNCTION

(71) Applicant: HIWELL Co., Ltd., Hwaseong-si (KR)

(72) Inventors: Yongnam KIM, Hwaseong-si (KR); Hyunmi KAM, Gunpo-si (KR); Minwoo KIM, Gunpo-si (KR); Minji KIM, Gunpo-si (KR)

(73) Assignee: HIWELL Co., Ltd., Hwaseong-si (KR)

(21) Appl. No.: 17/994,371

(22) Filed: Nov. 27, 2022

(30) Foreign Application Priority Data
Jul. 27, 2022 (KR) 10-2022-0092933

(51) Int. Cl. B01D 46/00; B01D 53/88; B01D 53/00; A61L 9/20

(52) U.S. Cl. CPC B01D 46/0028; B01D 46/0047; B01D 53/885; B01D 53/007; A61L 9/205; B01D 2255/802; B01D 2259/804; B01D 2255/20707; B01D 2279/65; A61L 2209/14

(57) ABSTRACT

Disclosed is a complex air purifier. The air purifier includes a device housing provided with an outdoor air inlet part and a purified air outlet, a filter unit provided in the device housing to purify air, a blowing fan provided in the device housing to enable the air introduced through the outdoor air inlet part to be filtered by the filter unit and the purified air to be discharged through the purified air outlet, and a sterilization unit provided in the device housing to sterilize the air purified by the filter unit, wherein the sterilization unit includes a photocatalytic sterilization unit configured to generate OH radicals through a photocatalytic reaction.

### BACKGROUND

### 1. Technical Field

The present disclosure relates to a complex air purifier having a sterilization function and, more particularly, to a complex air purifier capable of not only purifying dust and odors mixed in the air, but also very effectively removing viruses contained in the air, and at the same time, capable of providing an air shower to remove fine dust on skin and clothes, as well as to sterilize and deodorize various infectious bacteria, viruses, and fungi.

### 2. Related Art

In general, an air purifier can make the indoor environment pleasant by forcibly sucking air in a room, filtering out dust and odors mixed in the air, and blowing the filtered air back into the room.

Such an air purifier may include a blowing fan and a filter. Accordingly, the indoor air may be sucked into the air purifier through a suction port formed in the air purifier by the operation of the blowing fan. In addition, as the air sucked into the air purifier passes through the filter, various kinds of dust and foreign substances contained in the air may be filtered and discharged from an outlet of the air purifier.

However, the conventional air purifier simply focuses on the configuration that allows the filtered air to flow away, so a problem arises in that if the air contains a virus as in recent times, the air containing the virus may spread farther through the air purifier.

In addition, since the conventional air purifier is not equipped with a sterilization function capable of sterilizing the filtered air, there is a problem in that if the filtered air contains a virus, the virus may spread far in the indoor space as the filtered air is discharged.

In this regard, Korean Patent Application Laid-Open No. 10-2014-0087352 entitled “Air Purifier” (Patent Document 1) by the present applicant discloses an air purifier including a housing through which air flows and a filter arrangement disposed in an air distribution path. In the air purifier of Patent Document 1, the air introduced into the housing may be discharged from the housing again as the air is filtered by the filter.

However, in the air purifier of Patent Document 1, it is not recognized at all that air flows at a slow speed so that viruses, bacteria, and the like in the air can be removed. In other words, when such an air purifier as in Patent Document 1 is used, there is a problem in that viruses contained in air cannot be removed in the air purifier, and may spread far in the indoor space by a flow of air through the air purifier.

In addition, an air purifier disclosed in Korean Patent Application Laid-Open No. 10-2018-0070525 (Patent Document 2) by LG Electronics Co., Ltd. includes an air intake part, an air discharge part, an outer casing defining a space therein, a motor disposed in the space of the outer casing, a blower connected to the motor, and a filter disposed inside the outer casing.

However, in the air purifier disclosed in Patent Document 2, by the blower, air is circulated upward, filtered through the filter, and then directly discharged through the air discharge part without the virus and bacteria being removed from the air. As such, Patent Document 2 also does not recognize at all that air flows at a slow speed so that viruses, bacteria, etc. in the air can be removed. In other words, when such an air purifier as in Patent Document 2 is used, there is a problem in that the virus contained in the air may not be removed in the air purifier and then spread far in the indoor space by a flow of air through the air purifier.

### SUMMARY

Therefore, in order to solve the above-mentioned conventional problems, various embodiments of the present disclosure are directed to a complex air purifier capable of not only purifying dust and odors mixed in the air, but also very effectively removing viruses contained in the air.

In addition, various embodiments are directed to a complex air purifier capable of providing an air shower to remove fine dust on skin and clothes, as well as to sterilize and deodorize various infectious bacteria, viruses, and fungi.

The object of the present invention is not limited to the objects mentioned above, and other objectives not mentioned will be clearly understood by those skilled in the art from the following description.

In a first embodiment of the present disclosure, a complex air purifier including: a device housing provided with an outdoor air inlet part and a purified air outlet; a filter unit provided in the device housing and configured to purify air; a blowing fan provided in the device housing and configured to enable the air introduced through the outdoor air inlet part to be filtered by the filter unit and the purified air to be discharged through the purified air outlet; and a sterilization unit provided in the device housing and configured to sterilize the air purified by the filter unit, wherein the sterilization unit includes a photocatalytic sterilization unit generating OH radicals through a photocatalytic reaction.

In the complex air purifier, wherein the device housing has a truncated polygonal pyramid shape with a triangular or rectangular cross-section, and the filter unit is composed of at least one of a pre-filter made of a nonwoven fabric material, a dust collecting filter, and a deodorizing filter.

In the complex air purifier, wherein the driving fan is configured to produce an ascending air current or a descending air current, and the sterilization unit is provided on the downstream side of the air flow in the device housing.

In the complex air purifier, wherein the sterilization unit comprises: a casing section through which air is able to flow; and a hydroxyl (OH) radical-generation module provided around the casing section to produce OH radicals with a photocatalytic reaction.

In the complex air purifier, wherein the casing section is composed of a multiple stage of circular or polygonal unit casings stacked in a vertical direction, and the hydroxyl radical-generation module comprises: an OH radical-generation member formed by coating at least one of ceramic, zeolite, and activated carbon in the form of beads or pellets with a photocatalytic material; and an ultraviolet irradiation lamp provided to irradiate ultraviolet rays onto the OH radical-generation member.

In the complex air purifier, wherein the unit casings comprise filling unit casings, each filled with a photocatalyst member, and empty spatial unit casings, the filling unit casings and the spatial unit casings being alternately arranged.

In the complex air purifier, wherein the OH radical-generation member is composed of a bead-shaped member coated with a photocatalytic material of TiO₂, and the hydroxyl radical-generation module further comprises an OH radical-generation enhancing member comprising at least one of a reflective bead member made of a material capable of reflecting light and a transparent bead member made of a material capable of transmitting light.

The complex air purifier further comprises an air shower-blowing unit provided in the device housing to blow a sterilizing air in the form of an air shower, the air shower-blowing unit comprising: an air shower-blowing fan provided on a downstream side of a flow of air through the sterilization unit; an air shower outlet part formed on the device housing where the air shower-blowing fan is located; and an air shower on-off switch provided in the device housing to turn on/off the air shower-blowing fan.

The complex air purifier having the sterilization function according to the present disclosure has the following effects:

First, the present disclosure has an effect of not only purifying dust and odors mixed in the air, but also removing viruses contained in the air.

Second, the present disclosure provides a very excellent sterilization and deodorization effect by maximizing the generation of OH radicals through a specific structure.

Third, the present disclosure has the effect of sterilizing and deodorizing various infectious bacteria, viruses, and fungi, and at the same time, removing fine dust on the skin and clothes through the provision of an air shower containing OH radicals.

Fourth, the present disclosure has an effect of securing product competitiveness by implementing complex functions such as air cleaning, sterilization, deodorization, and an air shower.

The effects of the present invention are not limited to those mentioned above, and other problems not mentioned may be clearly understood by those skilled in the art from the following description.

### BRIEF DESCRIPTION OF THE DRAWINGS

FIG. 1 is a perspective view illustrating a complex air purifier having a sterilization function according to an embodiment of the present disclosure;

FIG. 2 is a front view illustrating the complex air purifier having the sterilization function according to the embodiment of the present disclosure;

FIG. 3 is a schematic front view illustrating internal components included in the complex air purifier having the sterilization function according to an embodiment of the present disclosure;

FIG. 4 is a front view illustrating a sterilization unit (photocatalytic sterilization unit) included in the complex air purifier having the sterilization function according to an embodiment of the present disclosure;

FIG. 5 is a plan view illustrating the sterilization unit (photocatalytic sterilization unit) included in the complex air purifier having the sterilization function according to the embodiment of the present disclosure;

FIG. 6 is a front cross-sectional view illustrating the sterilization unit (photocatalytic sterilization unit) included in the complex air purifier having the sterilization function according to the embodiment of the present disclosure;

FIG. 7 is a side cross-sectional view illustrating the sterilization unit (photocatalytic sterilization unit) included in the complex air purifier having the sterilization function according to the embodiment of the present disclosure;

FIG. 8 is a view illustrating a configuration of the sterilization unit (photocatalytic sterilization unit) included in the complex air purifier having the sterilization function according to an embodiment of the present disclosure; and

FIG. 9 is a view illustrating the operation of the complex air purifier having the sterilization function according to an embodiment of the present disclosure.

### DETAILED DESCRIPTION

Additional objects, features, and advantages of the present disclosure will be understood more clearly from the following detailed description and the accompanying drawings.

Prior to the detailed description of the present disclosure, the present disclosure may be variously modified and may have various embodiments, and it should be understood that examples to be described below and illustrated in the drawings is not intended to limit the present disclosure to specific embodiments and include all modifications, equivalents, and substitutes included in the spirit and technical range of the present disclosure.

It will be understood that when an element is referred to as being “connected” or “coupled” to another element, it can be directly connected or coupled to the other element or intervening elements may be present therebetween. In contrast, when an element is referred to as being “directly connected” or “directly coupled” to another element, there are no intervening elements therebetween.

The terminology used herein is for the purpose of describing particular embodiments only and is not intended to limit the disclosure. As used herein, the singular forms “a”, “an” and “the” include the plural forms as well, unless the context clearly indicates otherwise. It will be further understood that the terms “comprises”, “comprising,”, “includes” and/or “including”, when used herein, specify the presence of stated features, numbers, steps, operations, elements, components, or combinations thereof, but do not preclude the presence or addition of one or more other features, numbers, steps, operations, elements, components, or combinations thereof.

Furthermore, a term such as “ . . . section”, “ . . . unit”, and “ . . . module” described in this specification means a unit for processing at least one function or operation, and this may be implemented with hardware, software, or a combination of the hardware and the software.

Furthermore, in the following description with reference to the accompanying drawings, the same reference numerals are given to the same components and a redundant description thereof will be omitted. Detailed descriptions related to well-known functions or configurations will be ruled out in order not to unnecessarily obscure subject matters of the present disclosure.

Throughout the specification, when a step is “on” or “before” another step, this includes the same right not only when one step is in a direct time series relationship with another, but also when it is in an indirect time series relationship where the order of two steps can be changed, such as a mixing step after each step.

Hereinafter, a complex air purifier having a sterilization function according to a preferred embodiment of the present disclosure will be described in detail with reference to the accompanying drawings.

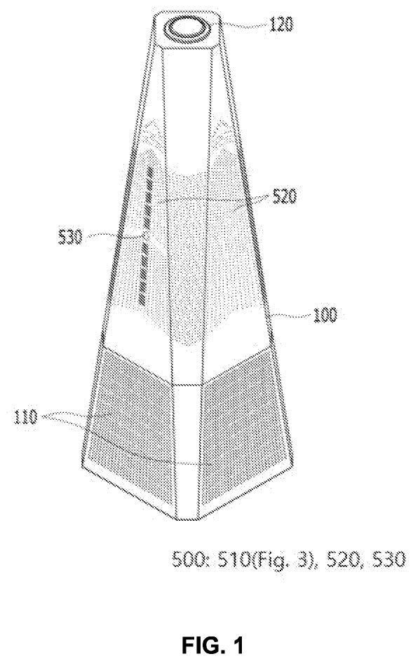

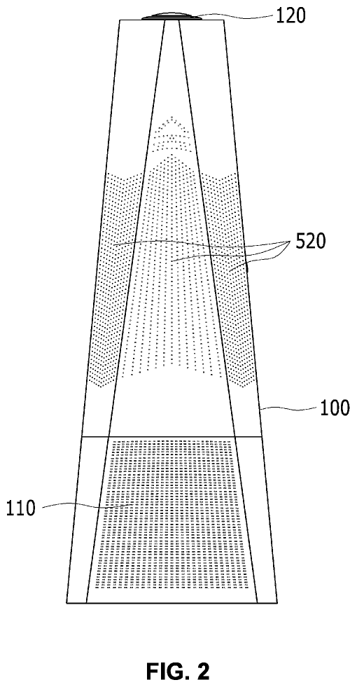

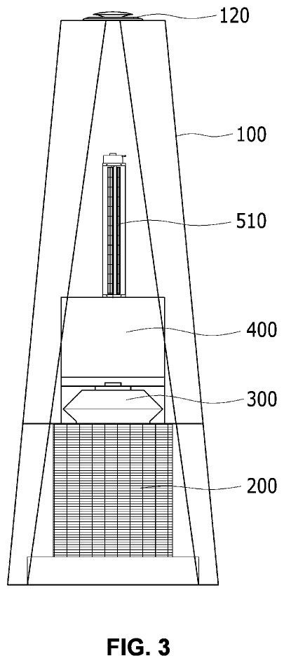

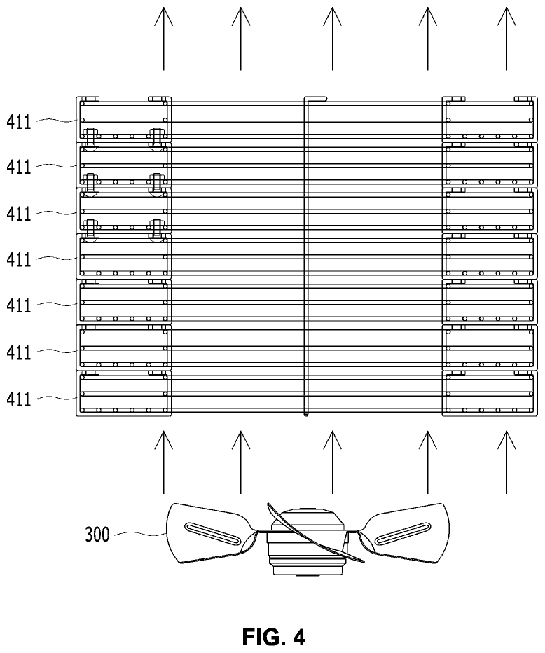

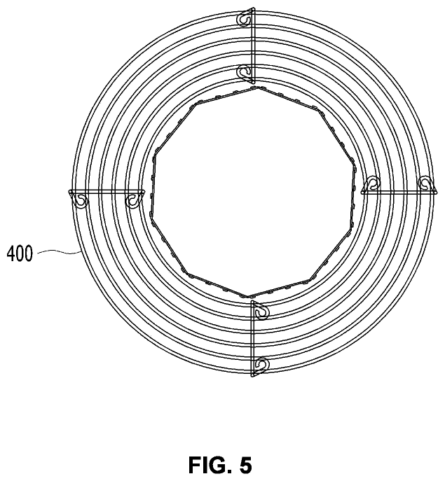

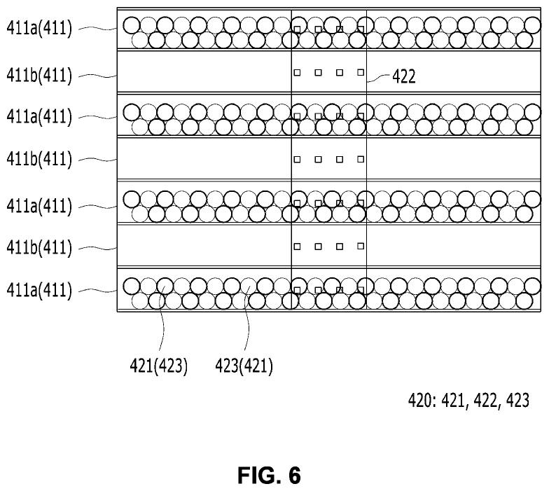

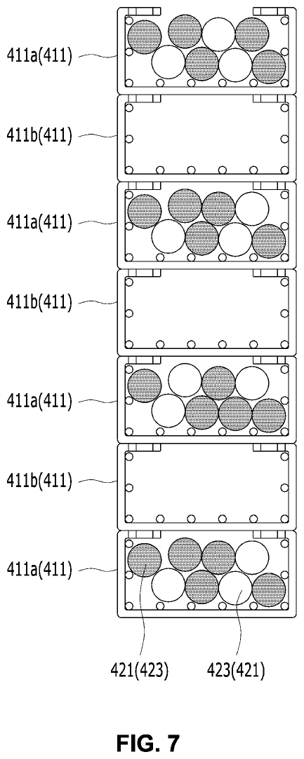

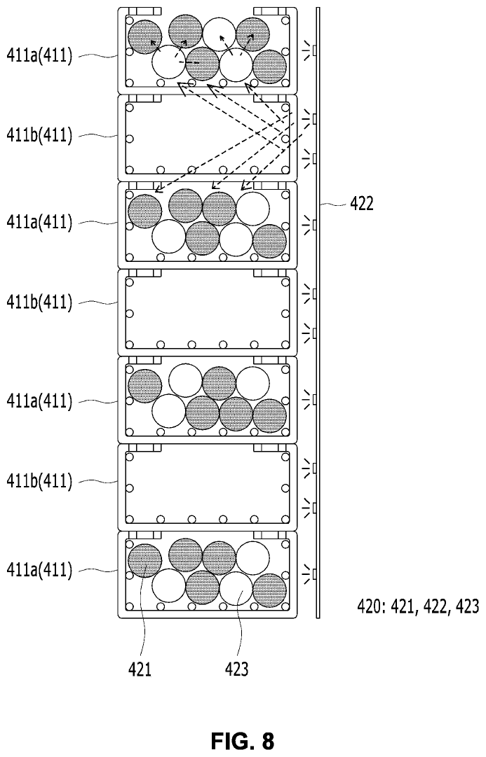

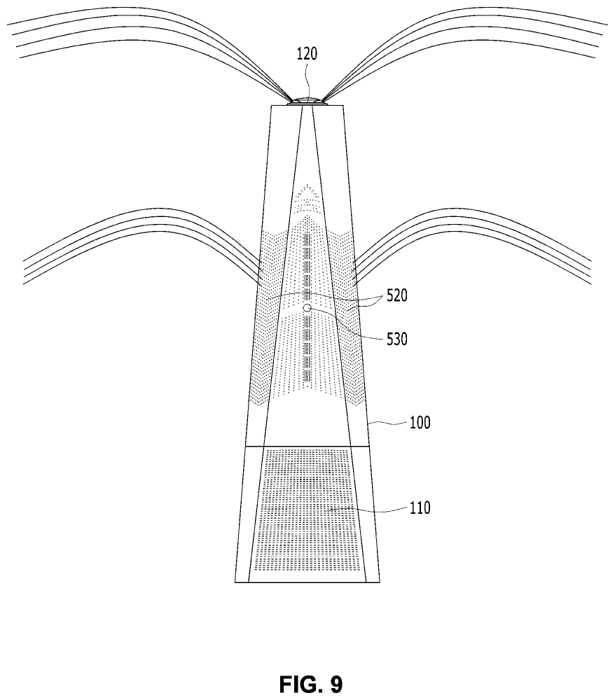

FIG. 1 is a perspective view illustrating a complex air purifier having a sterilization function according to an embodiment of the present disclosure; FIG. 2 is a front view illustrating the complex air purifier having the sterilization function according to the embodiment of the present disclosure; FIG. 3 is a schematic front view illustrating an internal components included in the complex air purifier having the sterilization function according to an embodiment of the present disclosure; FIG. 4 is a front view illustrating a sterilization unit (photocatalytic sterilization unit) included in the complex air purifier having the sterilization function according to an embodiment of the present disclosure; FIG. 5 is a plan view illustrating the sterilization unit (photocatalytic sterilization unit) included in the complex air purifier having the sterilization function according to the embodiment of the present disclosure; FIG. 6 is a front cross-sectional view illustrating the sterilization unit (photocatalytic sterilization unit) included in the complex air purifier having the sterilization function according to the embodiment of the present disclosure; FIG. 7 is a side cross-sectional view illustrating the sterilization unit (photocatalytic sterilization unit) included in the complex air purifier having the sterilization function according to the embodiment of the present disclosure; FIG. 8 is a view illustrating a configuration of the sterilization unit (photocatalytic sterilization unit) included in the complex air purifier having the sterilization function according to an embodiment of the present disclosure; and FIG. 9 is a view illustrating the operation of the complex air purifier having the sterilization function according to an embodiment of the present disclosure.

As illustrated in FIGS. 1 to 9, the complex air purifier having a sterilization function according to the present disclosure includes a device housing 100, a filter unit 200, a driving fan 300, and a sterilization unit 400. The complex air purifier may further include an air shower-blowing unit 500.

Specifically, as illustrated in FIGS. 1 to 9, in the complex air purifier, the device housing 100 is formed in a predetermined shape and has an outdoor air inlet part 110 on a lower side thereof, and a purified air outlet 120, which is provided on an upper end thereof so that the purified and sterilized air is discharged therethrough. The filter unit 200 is provided on a lower side in the device housing 100 to purify fine dust and/or odor. The driving fan 300 is provided on the filter unit 200 to suck and discharge the outdoor air to the outside, preferably forming an ascending air current. The sterilization unit 400 is provided above the driving fan 300 to sterilize the incoming air. In addition, the air shower-blowing unit 500 is further provided in the device housing 100 to blow the purified and sterilized air from the device housing 100 in the form of an air shower.

The device housing 100 is configured such that outdoor air is introduced from the outdoor air inlet part 110 on the lower side, and the purified and sterilized air is discharged through the purified air outlet 120 on the upper end thereof.

Specifically, the device housing 100 may be formed in a quadrangular truncated pyramid shape as illustrated in the drawings in an embodiment. Of course, the device housing 100 may be formed in a truncated cone shape with a circular cross section or a truncated polygonal pyramid shape such as a triangle or a pentagon. Here, the quadrangular truncated pyramid shaped device housing 100 may have a base portion with chamfered corners each having a predetermined width as illustrated in the drawing, or with rounded corners.

In addition, the outdoor air inlet part 110 formed on the lower side of the device housing 100 may be provided on at least one lateral surface (preferably all lateral surfaces) with a plurality of circular, polygonal, or slot-shaped holes through which air can be introduced. In the drawings, the outdoor air inlet part 110 of the device housing 100 is illustrated as being formed in a slot shape or a grill shape.

In addition, the device housing 100 is provided with a control panel. For example, the control panel may include a device on/off switch, a driving fan-output control switch, and the like.

As will be described below, the device housing 100 is provided on a middle portion thereof (i.e., on the side where the air shower-blowing fan is located) with an air shower outlet through which air that has been purified through the filter unit 200 and has been sterilized through the sterilization unit 400 is discharged. This will be described in detail below.

The filter unit 200 is the configuration that is provided on the lower side in the device housing 100 to purify fine dust and/or odor of outdoor air introduced by the driving operation of the driving fan 300.

The filter unit 200 may be composed of, for example, a HEPA filter member.

Preferably, the filter unit 200 may be composed of at least one of a pre-filter made of a non-woven fabric material capable of removing dust, pet hair, pollen, etc., a dust collecting filter capable of removing ultra-fine dust, etc., and a deodorizing filter capable of removing odors, sick house syndrome-causing substances, and harmful gases, etc.

The configuration and material of the filter unit 200 may employ well-known configuration and material, so a detailed description thereof will be omitted.

The driving fan 300 is a suction fan or a blowing fan that is provided on the filter unit 200 to suck and discharge the outdoor air to the outside.

In the present disclosure, the driving fan 300 is configured to form an ascending current of the air that has been purified through the filter unit 200 after being introduced from the outside, so that the air is blown toward the sterilization unit 400.

The sterilization unit 400 is the configuration that is provided above the driving fan 300 to sterilize the air that has been purified through the filter unit 200.

The sterilization unit 400 is composed of a photocatalytic sterilization unit.

Specifically, the sterilization unit 400 includes a casing section 410, through which air is able to flow, and a hydroxyl (OH) radical-generation module 420 that is provided around the casing section 410 to generate OH radicals through a photocatalytic reaction.

The casing section 410 is composed of a multiple stage of circular or polygonal unit casings 411 stacked in a vertical direction, wherein each of the unit casing has a central through-portion.

In the casing section 410, the unit casings include photocatalyst-filling unit casings 411a and empty spatial unit casings 411b, which are alternately arranged, wherein each of the photocatalyst-filling unit casing is filled with a photocatalyst member, which is a component of the hydroxyl radical-generation module 420 to be described below.

The hydroxyl radical-generation module 420 includes an OH radical-generation member 421 formed by coating at least one of ceramic, zeolite, and activated carbon in the form of beads or pellets with a photocatalytic material, and an ultraviolet irradiation lamp 422 provided to irradiate ultraviolet rays onto the OH radical-generation member 421.

The OH radical-generation member 421 is a bead-shaped member coated with a photocatalytic material of TiO₂.

The ultraviolet irradiation lamp 422 is provided at a position where the ultraviolet irradiation lamp can irradiate ultraviolet rays toward the OH radical-generation member 421 on the side of the casing section 410.

The ultraviolet irradiation lamp 422 may be composed of a UV-A lamp irradiating ultraviolet rays of a wavelength of 320 to 400 nm, and in addition, a UV-C lamp irradiating ultraviolet rays of a wavelength of 200 to 280 nm.

On the other hand, the hydroxyl radical-generation module 420 may further include an OH radical-generation enhancing member 423, in addition to the OH radical-generation member 421, wherein the OH radical-generation enhancing member 423 includes a reflective bead member made of a material capable of reflecting light (ultraviolet rays) and/or a transparent bead member made of a material capable of transmitting light.

Here, the OH radical-generation enhancing member 423 may be partially or entirely coated with a photocatalytic material of TiO₂.

Here, as illustrated in FIG. 8, the unit casings 411 of the casing section 410 are composed of the filling unit casings 411a which is filled with the OH radical-generation member 421 and the OH radical-generation enhancing member 423, and the empty spatial unit casings 411b arranged between the filling unit casings 411a. This not only makes the air flow more smoothly, but also enables the ultraviolet irradiation lamp 422 to irradiate the ultraviolet rays onto the OH radical-generation member 421 and the OH radical-generation enhancing member 423 more evenly through the empty spatial unit casings 411b. At the same time, this also enables a photocatalytic reaction to occur more actively in the filling unit casings 411a due to the OH radical-generation enhancing member 423 that is provided in the filling unit casings 411a so that light is reflected therefrom or transmitted therethrough.

The inventors of the present application confirmed that, through an experiment, when the OH radical-generation enhancing member 423 was provided together with the OH radical-generation member 421, the generation of the OH radicals was increased by 35% or more.

The capacity of the air purifier may be determined by adjusting the number of unit casings 411 (411a and 411b) to be stacked.

On the other hand, the complex air purifier having a sterilization function according to the present disclosure may further include an air shower-blowing unit 500 that is provided in the device housing 100 to blow the purified and sterilized air from the device housing 100 in the form of an air shower.

The air shower-blowing unit 500 includes an air shower-blowing fan 510 on the upper side of the sterilization unit 400, an air shower outlet part 520 formed on the device housing 100 where the air shower-blowing fan 510 is located to discharge the air blown by the blowing fan 510 in the form of an air shower, and an air shower on-off switch provided in the device housing 100 to turn on/off the air shower-blowing fan 510.

The blowing fan 510 may be configured as a blowing fan that blows air while rotating at 360°, or as a blowing fan that blows air while horizontally rotating at a predetermined range of angle, for example, 180°.

The air shower outlet part 520 may be provided on at least one lateral surface (preferably all lateral surfaces) of the device housing 100 with a plurality of circular, polygonal, or slot-shaped holes through which air can be blown. In the drawings, the air shower outlet part 520 is illustrated as being formed as a plurality of holes.

In addition, the device housing 100 is provided with a control panel. For example, the control panel may include a device on/off switch, a driving fan-output control switch, and the like.

On the other hand, in the above description, the arrangement relationship between the filter unit 200, the driving fan 300, and the sterilization unit 400 is such that air that is purified through the filter unit 200 and is contained with OH radicals generated through the sterilization unit 400 is discharged from the bottom, where the air shower-blowing unit 500 may be configured on the downstream side of the sterilizing air.

As described before, to the complex air purifier having a sterilization function according to the present disclosure has an effect of not only purifying dust and odors mixed in the air, but also removing viruses contained in the air, as well as providing very excellent sterilization and deodorization features by maximizing the generation of radicals (OH radicals) through specific structures and configurations.

In addition, according to the present disclosure, it is possible to sterilize and deodorize various infectious bacteria, viruses, and molds while removing fine dust on the skin and clothes through an air shower containing OH radicals, which implements complex functions such as air cleaning, sterilization, deodorization, and an air shower, thereby providing the effect of securing product competitiveness.

While various embodiments have been described above, it will be understood to those skilled in the art that the embodiments described are by way of example only. Accordingly, the disclosure described herein should not be limited based on the described embodiments.

The embodiment and the accompanying drawings described in the present specification are merely intended to describe a part of the technical spirit included in the present disclosure. Therefore, since the embodiment disclosed in the present specification is not intended to limit the technical spirit of the present disclosure but to explain the technical spirit of the present disclosure, it is obvious that the scope of the technical spirit of the present disclosure is not limited by such an embodiment. Modifications and specific embodiments easily inferred by those skilled in the art within the scope of the technical spirit included in the specification and the drawings of the present disclosure should be construed as being included in the scope of the present disclosure.

What is claimed is:

1. A complex air purifier comprising:
a device housing provided with an outdoor air inlet part and a purified air outlet;
a filter unit provided in the device housing to purify air;
a blowing fan provided in the device housing to enable the air introduced through the outdoor air inlet part to be filtered by the filter unit and the purified air to be discharged through the purified air outlet; and
a sterilization unit provided in the device housing to sterilize the air purified by the filter unit, wherein the sterilization unit includes a photocatalytic sterilization unit configured to generate OH radicals through a photocatalytic reaction.

2. The complex air purifier according to claim 1, wherein the device housing has a truncated polygonal pyramid shape with a triangular or rectangular cross-section, and the filter unit is composed of at least one of a pre-filter made of a nonwoven fabric material, a dust collecting filter, and a deodorizing filter.

3. The complex air purifier according to claim 1, wherein the driving fan is configured to produce an ascending air current or a descending air current, and the sterilization unit is provided on the downstream side of the air flow in the device housing.

4. The complex air purifier according to claim 1, wherein the sterilization unit comprises:
a casing section through which air is able to flow; and
a hydroxyl (OH) radical-generation module provided around the casing section to produce OH radicals with a photocatalytic reaction.

5. The complex air purifier according to claim 4, wherein the casing section is composed of a multiple stage of circular or polygonal unit casings stacked in a vertical direction, and
the hydroxyl radical-generation module comprises: an OH radical-generation member formed by coating at least one of ceramic, zeolite, and activated carbon in the form of beads or pellets with a photocatalytic material; and an ultraviolet irradiation lamp provided to irradiate ultraviolet rays onto the OH radical-generation member.

6. The complex air purifier according to claim 5, wherein the unit casings comprise filling unit casings, each filled with a photocatalyst member, and empty spatial unit casings, the filling unit casings and the spatial unit casings being alternately arranged.

7. The complex air purifier according to claim 5, wherein the OH radical-generation member is composed of a bead-shaped member coated with a photocatalytic material of TiO₂, and
the hydroxyl radical-generation module further comprises an OH radical-generation enhancing member comprising at least one of a reflective bead member made of a material capable of reflecting light and a transparent bead member made of a material capable of transmitting light.

8. The complex air purifier according to claim 1, further comprises an air shower-blowing unit provided in the device housing to blow a sterilizing air in the form of an air shower, the air shower-blowing unit comprising:
an air shower-blowing fan provided on a downstream side of a flow of air through the sterilization unit;
an air shower outlet part formed on the device housing where the air shower-blowing fan is located; and
an air shower on-off switch provided in the device housing to turn on/off the air shower-blowing fan.

9. The complex air purifier according to claim 6, wherein the OH radical-generation member is composed of a bead-shaped member coated with a photocatalytic material of TiO₂, and
the hydroxyl radical-generation module further comprises an OH radical-generation enhancing member comprising at least one of a reflective bead member made of a material capable of reflecting light and a transparent bead member made of a material capable of transmitting light.
